(19) United States
(12) Patent Application Publication
(10) Pub. No.: US 2023/0414400 A1
(43) Pub. Date: Dec. 28, 2023

(54) THUMB SUCKING DETERRENT SYSTEM

(71) Applicants: Kinan Muhsin Salloum, Damascus (SY); Fernanda Oliveira Duarte, Guarulhos – SP (BR)

(72) Inventors: Kinan Muhsin Salloum, Damascus (SY); Fernanda Oliveira Duarte, Guarulhos – SP (BR)

(21) Appl. No.: 18/275,008

(22) PCT Filed: May 5, 2021
(86) PCT No.: PCT/IB2021/053773

§ 371 Date: Jul. 30, 2023

(51) Int. Cl. A61F 5/50; H04B 5/00

(52) U.S. Cl. CPC A61F 5/50; H04B 5/0062; G08B 3/00

(57) ABSTRACT

A thumb sucking deterrent system comprising a tag configured to be removably fitted inside mouth of a child. The tag comprises a tag antenna which is anatomically shaped to correspond to sections of a child's dentition and an integrated circuit chip which is conductively mounted to the tag antenna. A wearable tag reader adapted to be worn on a part of a forearm of the child is provided. The tag reader comprises a transceiver module, a control module and an alarm module. The alarm module is configured to initiate alert actions when the control module determines an actual distance between the tag and the tag reader to be within a predefined range based on a reply signal generated by the tag in response to a first interrogation signal generated by the transceiver module. The distal ends of the tag antenna are structured to be secured to teeth of the child.

### FIELD OF THE INVENTION

The present invention in general is related to devices for deterring finger-sucking habits in children. More particularly, the present invention is related to a smart system for deterring finger-sucking habits in children.

### BACKGROUND OF THE INVENTION

One of the unhealthy and displeasing habits frequently adopted by children is finger sucking or thumb sucking. These habits, although, are extensions of children's inborn inclination to suck, some children take the habit well beyond their age when they are weaned from bottle-feeding or breast-feeding. The term “thumb sucking” is hereinafter used to denote sucking one's index finger, thumb or any other manual digit except where the context of usage indicates otherwise.

Thumb sucking is not only unpleasant to look at but, also can have side effects injurious to a person's health. For example, there can be germs or dirt on the thumb sucker's hands and these can be harmful if ingested. When the child begins teething and thumb sucking continues, the continuous presence of a thumb between the gums can divert the arriving teeth from their usual positions in the jaw. The result is crooked teeth when the thumb sucking problem continues through the coming of permanent teeth. This can be dealt with only by orthodontic treatment which is not only time-consuming but also very expensive, e.g. braces. Continuous thumb sucking at this stage also can damage the skin on the thumb, leading to infection. Additionally, thumb sucking can hurt a child emotionally if the child is rebuked by adults or teased by his or her peers for thumb sucking.

Numerous attempts to overcome the thumb sucking problem are known in the art as thumb sucking is a recognized problem. For example, there are different dental appliances which fit in the roof of the thumb sucker's mouth. Such dental appliances have prongs or other protrusions extending downwardly from the roof of the mouth so as to prevent the thumb sucker from inserting one's thumb into the mouth. These kinds of devices affect the speech of the wearer and can be very annoying, and such devices as a result are seldom used.

Another technique for preventing thumb sucking includes applying various bitter-tasting liquids to the thumb of the thumb sucker. The intention here is that the thumb sucker, associating an unpleasant taste or a bitter taste with the act of thumb sucking, will stop sucking his or her thumb. In reality, the result is not that fruitful as the thumb sucker washes his or her hands, removing the bitter taste resumes thumb sucking.

Different other types of anti-thumb sucking devices for wearing on the thumb sucker's hand are also known in the art. Such anti-thumb sucking devices generally have attempted to stop thumb sucking either by causing the wearer discomfort when trying to insert thumb into mouth, or by preventing the thumb sucker from forming an airtight seal around the thumb and thus stopping the sucking action desired by the child.

Other devices, known in the art, intended to prevent thumb sucking include overwrap for fingers and hands, glove for preventing thumb sucking etc. Such devices are rarely delightful to the parents or to the child because of their functional design. Thus, it is often very difficult to convince parents to encourage the wearing of these devices by the child. Ideally, thumb sucking preventative devices should include features which are comfortable to the child, such that the child not only bears its use, but happily wear it most of the time during treatment.

Thus, there exists a need for a system which overcomes the above-mentioned disadvantages associated with devices made for preventing thumb-sucking habits in children.

### OBJECTS OF THE INVENTION

An object of the present invention is to provide a system for discouraging thumb sucking habits in children.

Yet another object of the present invention is to provide an inexpensive thumb-sucking deterrent device.

A further object of the present invention is to provide a thumb sucking deterrent device that does not restrict natural movements of the hands or fingers of a child.

A still further object of the present invention to provide a thumb sucking deterrent system which enables sending of an alert to a remotely located computing device in real time against every thumb-sucking attempt made by a child.

Another object of the present invention is to provide a thumb-sucking deterrent device which is configurable in terms of detection criteria against thumb-sucking attempts.

Yet another object of the present invention is to provide a system that enables monitoring of activities related to thumb-sucking habit of a child.

Other objects and further scope of applicability of the present invention will become apparent from the detailed description given hereinafter. It should be understood, however, that the detailed description and specific examples, while indicating preferred embodiments of the invention, are given by way of illustration only, since various changes and modifications within the spirit and scope of the invention will become apparent to those skilled in the art from this detailed description.

### SUMMARY OF THE INVENTION

The following presents a simplified summary in order to provide a basic understanding of some aspects of the disclosed invention. This summary is not an extensive overview, and it is not intended to identify key/critical elements or to delineate the scope thereof. Its sole purpose is to present some concepts in a simplified form as a prelude to the more detailed description that is presented later.

The present invention is directed to a system which deters thumb sucking habit in children. The system comprises an RFID tag configured to be temporarily wearable inside the mouth of a child, an RFID tag reader wearable on the forearm of the child and a computing device communicatively connected to the RFID tag reader. The RFID tag, also referred hereinafter as wearable tag, is wearable inside the mouth of a child and comprises a tag antenna and an integrated circuit chip electrically bonded to the tag antenna. The dimensions and shape of the tag antenna and the chip are customized to fit inside the mouth of a child along the lingual or facial sides of teeth. The distal ends of the tag antenna are elongated members which are structured to be anchored to one or more teeth while holding a frontal section of it at a desired place inside the mouth.

The RFID tag reader, hereinafter also referred to as wearable tag reader, comprises a transceiver module, a control module, a network module and an alarm module. The transceiver module generates and transmits a modulated interrogational radio frequency signal. The tag antenna, particularly a frontal section of the tag antenna, that is designed as a conventional RFID tag antenna, receives this interrogation signal and forms an electric and magnetic field. The chip of the wearable tag draws this electrical power and transmits a reply radio frequency signal back to the wearable tag reader through the tag antenna. The control module continuously calculates the actual distance between the wearable tag reader and the wearable tag based on the reply signal received from the wearable tag. A predefined range of distance is set with the control module so that whenever the calculated actual distance becomes less than the predefined range of distance then an alert action is initiated by the wearable tag reader. Therefore, since for sucking of a thumb/finger a child must bring its hand(s) close to its mouth, every time the child tries to suck a thumb the distance between the wearable tag reader and the wearable tag worn by the child becomes less than the predefined distance and the buzzer provided on the wearable tag reader goes off. The sound of the alarm/buzzer acts as a deterrent against thumb sucking habit. A notification is also sent to the computing device which is connected to the wearable tag reader every time the alarm goes off.

### BRIEF DESCRIPTION OF THE DRAWINGS

In order to describe the manner in which features and other aspects of the present disclosure can be obtained, a more particular description of certain subject matter will be rendered by reference to specific embodiments which are illustrated in the appended drawings. Understanding that these drawings depict only typical embodiments and are not therefore to be considered to be limiting in scope, nor drawn to scale for all embodiments, various embodiments will be described and explained with additional specificity and detail through the use of the accompanying drawings in which:

FIG. 1 illustrates a thumb sucking habit of a child;

FIG. 2 illustrates a general architecture of a thumb sucking deterrent system that operates in accordance with an embodiment of the present invention;

FIG. 3 illustrates interaction between a wearable tag and a wearable tag reader in accordance with an embodiment of the present invention;

FIG. 4 illustrates a wearable tag implanted inside the mouth of a child/person in accordance with an embodiment of the present invention;

FIG. 5A illustrates a child wearing the wearable tag and the wearable tag reader of the present invention;

FIG. 5B illustrates a child wearing the wearable tag and the wearable tag reader of the present invention bringing its hand close to its mouth while trying to suck its thumb;

FIG. 6 illustrates another embodiment of the wearable tag implanted inside the mouth of a child/person in accordance with an embodiment of the present invention;

FIG. 7 illustrates a general architecture of a thumb sucking deterrent system having an additional slave wearable tag reader that operates in accordance with an embodiment of the present invention; and

FIG. 8 illustrates interactions among a wearable tag, a wearable tag reader and a slave wearable tag reader in accordance with an embodiment of the present invention.

### DETAILED DESCRIPTION OF THE INVENTION

Embodiments of the invention are discussed below with reference to the figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures/computer architectures described herein are to be understood also to refer to functional equivalents of such structures. The present invention will now be described in detail with reference to embodiments thereof as illustrated in the accompanying drawings.

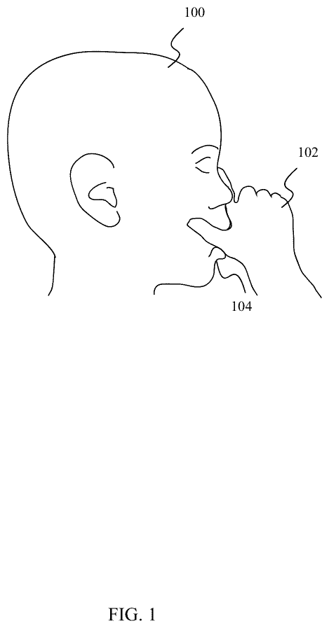

FIG. 1 is an exemplary illustration of a child 100 with its hand 102 close to its mouth 104 for sucking its thumb.

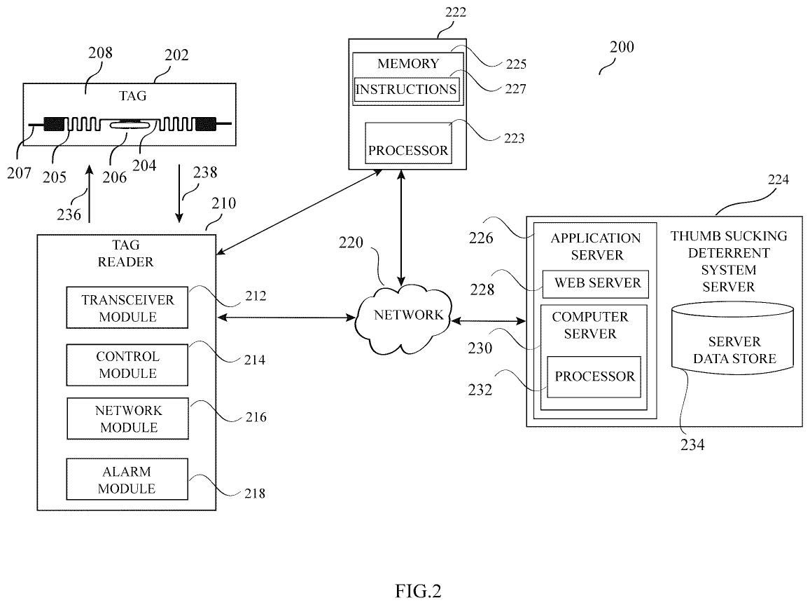

Reference to FIG. 2, the thumb sucking deterrent system 200 of the present invention comprises a wearable tag 202, one or more wearable tag readers 210, one or more computing devices 222 and a remote thumb sucking deterrent system server 224.

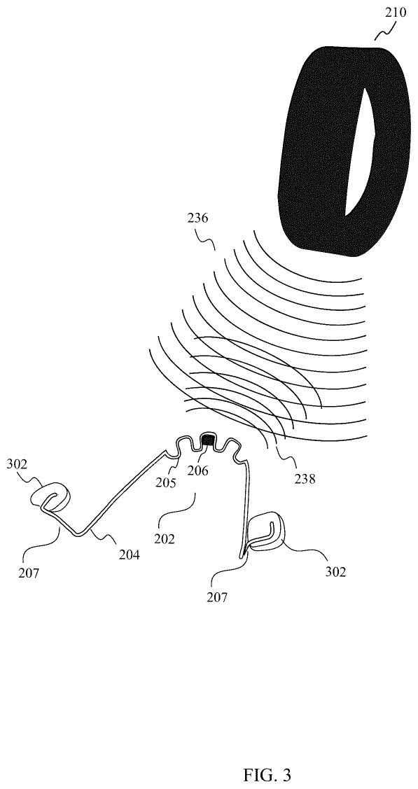

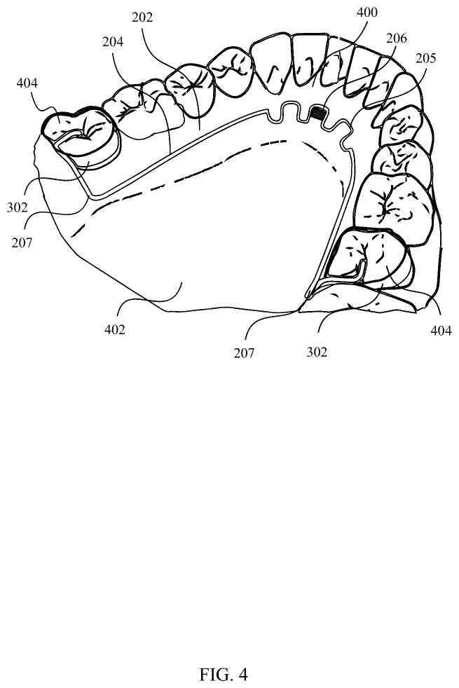

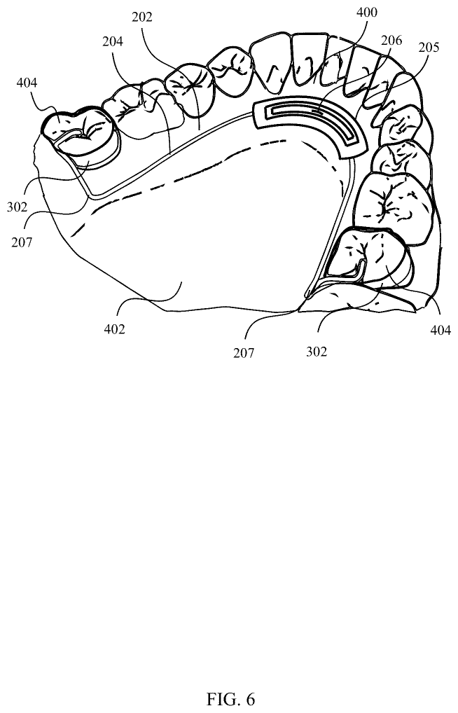

Reference to FIGS. 2, 3 and 4, the wearable tag 202, which acts as a radio frequency identification tag (RFID tag), comprises a tag antenna 204 and an integrated circuit chip 206. The wearable tag 202 is adapted to be worn removably inside mouth of a child/person. As shown in FIGS. 3 and 4, the tag antenna 204 is anatomically shaped to correspond to one or more sections of a person's jaw area 400 and palate area 402 so that the tag antenna 204 can be positioned on dentition of the upper jaw or the lower jaw of a child/person as being shown in the drawings. The dimensions of the tag antenna 204 can be customized according to the dentition of a person. The tag antenna 204 comprises distal ends 207 which are elongated members structured to be secured to one or more teeth 404 of a person, and a specifically designed frontal section 205 capable of receiving and transmitting radio waves. For removably securing/fixing the wearable tag 202 inside the mouth of a person, the distal ends 207 are anchored to one or more teeth 404 of the person. Additionally, adjustable bands 302 disposed at the distal ends 207 can be strapped around teeth to secure the wearable tag 202. The frontal section 205 is configured as an RFID tag antenna. The frontal section 205 can have conventional RFID tag antenna designs such as dipole, loop, spiral, patch, slot, or meander designs while conforming to the desired form, fit, and functional performance. For example, in the embodiment of the present invention shown in FIGS. 3 and 4, the frontal section 205 of the tag antenna 204 is designed as a dipole antenna whereas, the frontal section 205 of the embodiment shown in FIG. 6 is designed as a loop type antenna. The integrated circuit chip/RFID chip 206 may be conductively/electrically bonded to the tag antenna 204, preferably to the frontal section 205 by soldering or by any other suitable method. The integrated circuit chip 206 facilitates the radio transmission and receiver functions of the wearable tag 202. The chip 206 also acts as data storage for storing data such as unique identification code etc. It is to be understood that, although the wearable tag 202 is shown fitted on the lingual side of teeth, it can be designed to be fitted on the facial side of the teeth also. The entire tag antenna 204 or a section of it (the frontal section 205, for example) along with the chip 206 is provided with a water resistant coating 208 to electrically insulate it from an outer environment.

Reference to FIGS. 2 and 3, the wearable tag reader 210 is adapted to be removably worn on the hand or forearm of a person, especially on the wrist area of a forearm. The wearable tag reader 210 comprises a transceiver module 212, a control module 214, a network module 216 and an alarm module 218. The transceiver module 212 can generate and transmit an electromagnetic interrogation pulse/signal 236 and receive a reply signal/pulse 238 from a nearby wearable tag 202. The control module 214 having a processor is configured to receive computer program instructions which, when executed by the control module 214 in general or by the processor in particular, controls the transceiver module 212, network module 216 and alarm module 218 for interrogation functionalities, analysis and detection of proximity of wearable tags 202, for interacting with external device over a network and for generating one or more alert actions. The control module 214 is provided with a memory which may comprise any computer-readable storage media readable by the processor of the control module 214 and capable of storing software such as the program instructions.

The computing device 222 comprises a processor 223 and a device memory 225. The device memory 225 may comprise any computer-readable storage media readable by the processor 223 and capable of storing software such as program instructions 227 for operating the wearable tag reader 210. In the present example, the computer program instructions 227 may be a mobile application or “mobile app” that may be downloaded and operably installed in a computing device using methods known in the art. This mobile app, also referred to herein as thumb sucking deterrent app, enables a user to do various tasks related to activation, operation and control of the thumb sucking deterrent system. Examples of computing device may include, but not limited to mobile devices, tablets, hand-held or laptop devices, smart phones, personal digital assistants or any other similar devices.

Examples of device memory include random access memory (RAM), read only memory (ROM), magnetic disks, optical disks, write-once-read-many disks, CDs, DVDs, flash memory, solid state memory or any other suitable storage media. Certain implementations may involve either or both virtual memory and non-virtual memory.

The network module 216 may enable communication between one or more devices. In a non-limiting, a network module 216 may enable communication between a computing device 222 and the wearable tag reader 210, between a remote server such as 224 and the wearable tag reader 210 as shown in FIG. 2.

In the present embodiment, computing device 222 may use a GUI module to provide a user interface on the computing device 222. The GUI module enables a user to view the activities related to operation of the thumb sucking deterrent system.

The process of providing deterrence to thumb sucking involving the present invention may happen under at least partial control and/or in communication with a remote server as shown in FIG. 2. The remote server i.e. the thumb sucking deterrent system server 224 communicates with the computing device 222 over the network 220. As used herein, the term “network” generally refers to any collection of distinct networks working together to appear as a single network to a user. The term also refers to the so-called world wide “network of networks” or Internet which is connected to each other using the Internet protocol (IP) and other similar protocols. As described herein, the exemplary public network 220 of FIG. 2 is for descriptive purposes only and it may be wired or wireless. Although, the description may refer to terms commonly used in describing particular public networks such as the Internet, the description and concepts equally apply to other public and private computer networks, including WiFi, Bluetooth etc. The inventive idea of the present invention is applicable for all existing cellular network topologies or respective communication standards, in particular GSM, UMTS/HSPA, LTE and future standards.

The thumb sucking deterrent system server 224 includes an application server or executing unit 226 and a data store 234. The application server or executing unit 226 comprises a web server 228 and a computer server 230 that serves as the application layer of the present invention. The computer server 230 may comprise one or more physical processors 232. The data store 234 is or includes a non-transitory computer-readable storage medium. The data store 234 may store computer program instructions to operate the thumb sucking deterrent system server 224.

The Web server 228 is a system that sends out Web pages containing electronic data files in response to Hypertext Transfer Protocol (HTTP) requests or similar protocol requests from remote browsers (i.e. browsers installed in the computing device) or in response to similar requests made through a mobile app or mobile application of the present invention installed on a computing device. The web server 228 can communicate with the mobile app of the present invention, with a firmware or software installed on a network appliance and/or with a web browser installed on a computing device 222 to provide the user interface required for the thumb sucking deterrent system 200.

Reference to FIGS. 2 and 3, the wearable tag reader 210 is configured to determine distance between the wearable tag reader 210 and the wearable tag 202. The transceiver module 212 of the wearable tag reader 210 generates and transmits a modulated interrogational radio frequency signal 236, hereinafter referred to as first interrogation signal 236, which the tag antenna 204 receives and forms an electric and magnetic field. On receiving the electrical power the chip 206 transmits a reply radio frequency signal 238, hereinafter referred to as reply signal 238, modulated with data, back to the wearable tag reader 210. The reply signal 238 may also include a unique code which has been assigned to each wearable tag 202 and stored by the chip 206.

The control module 214 uses various methods to measure an actual distance between a wearable tag 202 and a wearable tag reader 210 and compare it against a predefined range of distance. If the actual distance is found to be less than the predefined range of distance then the alarm module 218 initiates one or more alert actions.

In one embodiment, to measure the distance between the wearable tag 202 and wearable tag reader 210, the control module 214 calculates the difference between the time the first interrogation signal 236 is sent and the time the reply signal 238 is received by the transceiver module 212. In another embodiment, the transceiver module 212 may generate a plurality of first interrogation signals 236 within an interrogation range. The characteristics of the received reply signals 238 are then analyzed by the control module 214 to determine the distance between the wearable tag 202 and wearable tag reader 210. An example could be use of Received Signal Strength Indicator (RSSI) to measure the distance. The control module 214 measures the RSSI of a reply signal 238 and calculates the distance between the wearable tag 202 and wearable tag reader 210. Another option is to make the transceiver module 212 generate the first interrogation signals 236 at differing frequencies. The differing frequencies will produce differing phases, and the differing frequencies and phases of the reply signals 238 are used by the control module 214 to compute the distance between the wearable tag 202 and wearable tag reader 210. In all the cases, the wearable tag reader 210 can identify a wearable tag 202 by the unique code assigned to each wearable tag 202 which the chip 206 stores.

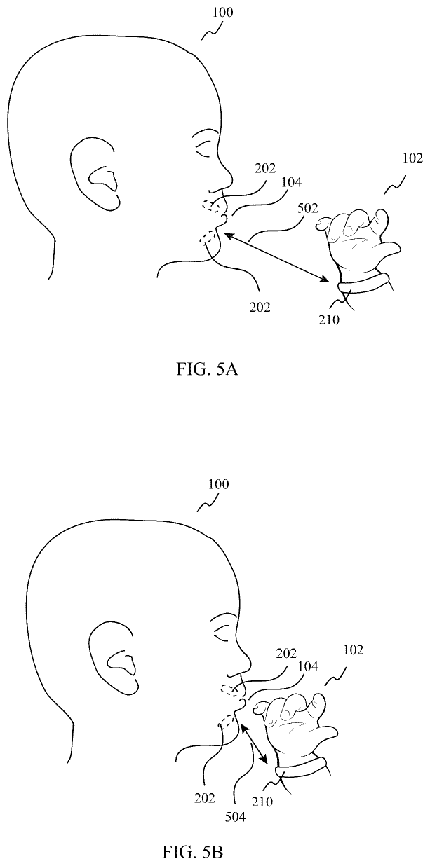

For use, the wearable tag 202 is fitted inside the mouth of a child who has thumb/finger sucking habit and the wearable tag reader 210 is worn on the forearm, especially in the wrist region, of the child as shown in FIGS. 5A and 5B. The settings of the wearable tag reader 210 in terms of setting of characteristics (frequency, strength etc., for example) of the generated first interrogation signals 236, setting of a predefined range for distance measurement, analysis of the reply signals 238, communication of the network module 216 with an external computing device 222 or with a remote server 224, and initiation of alert actions by the alarm module 218 can all be controlled by giving one or more instructions to the control module 214. Such instructions can be given to the control module 214 using a built-in user interface provided on the wearable tag reader 210 or through a graphical user interface (GUI) provided on a communicatively connected computing device 222 or from a remote server 224.

The predefined range is set in such a way that, when the hand 102 of the child 100 is at a reasonable distance away from its mouth 104, for example at a distance of 1 foot as indicated by reference numeral 502 shown in FIG. 5A, the wearable tag reader 210 does not initiate any alert action. To suck its thumb/finger the child 100 has to bring its hand 102 close to its mouth 104. The control module 214 continuously keeps on calculating the actual distance between wearable tag reader 210 worn by the child 100 and the wearable tag 202 fitted inside the mouth 104 of the child 100. So, as shown in FIG. 5B, as the hand 102 comes close to the mouth 104, the actual distance 504 between the wearable tag reader 210 worn on the hand 102 and the wearable tag 202 fitted inside the mouth 104 becomes less than the predefined range (which is 1 foot in the present example). As soon as the control module 214 determines that the actual distance 504 is less than the predefined distance/range 502 the alarm module 218 is instructed to initiate one or more alert actions.

One of the alert actions initiated by the alarm module is an audible alarm emitted by a speaker included in said alarm module and/or a visual alarm emitted by a light source disposed in said wearable tag reader. So, with the implementation of the present invention, every time the child brings its hands close to its mouth for sucking a thumb/finger/digit, the audible alarm buzzes. This audible alarm generated by the wearable tag reader 210 will scare the child and is likely to act as a deterrent against thumb/finger sucking. In another embodiment, a notification is sent to the computing device 222 and/or to the thumb sucking deterrent system server 224 over the network 220 against every alert action. Such notifications can make a person taking care of the child aware of the attempts being made by child for thumb sucking. The notifications also get stored in the computing device 222 or in the remote thumb sucking deterrent system server 224 so that those can be reviewed for analyzing thumb sucking habit of the child.

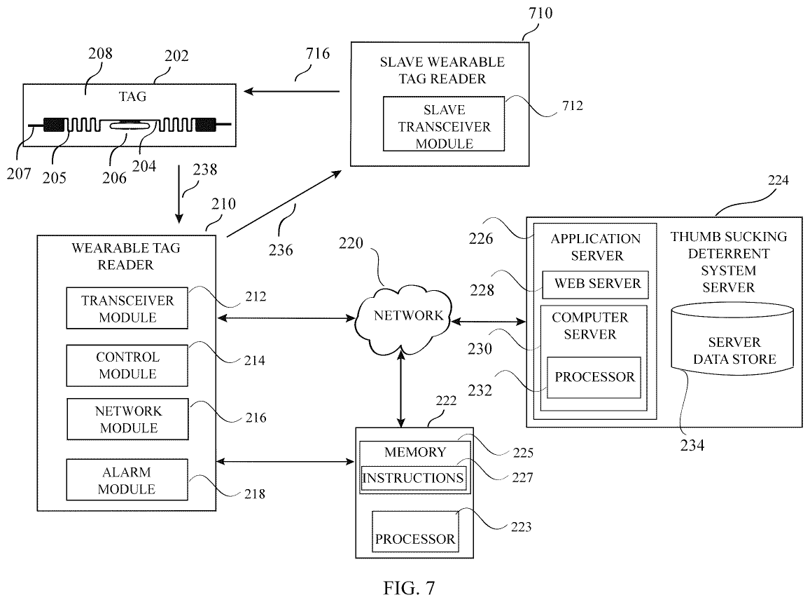

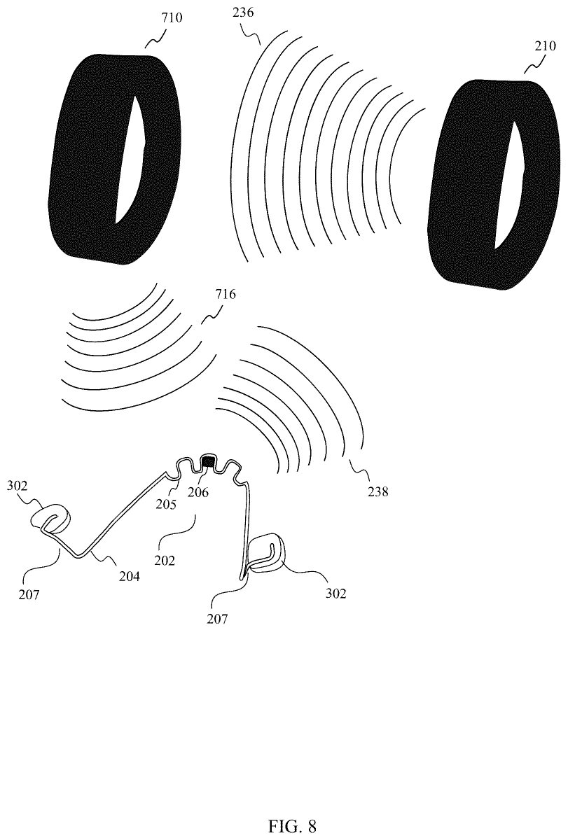

If a child has a habit of sucking thumbs/fingers of its both hands then it is required that the alert actions are initiated whenever either of the hands comes closer to the mouth of the child. In such cases the wearable tag reader 210 can be worn on both the hands of a child. However, reference to FIGS. 7 and 8, the same desired deterrence can be achieved if a slave wearable tag reader 710 is worn in one hand of the child and a wearable tag reader 210 being worn on the other hand acts as a master.

Reference to FIGS. 7 and 8 the slave wearable tag reader 710 is adapted to be removably worn on the forearm of a person, especially on the wrist area of a forearm. The slave wearable tag reader 710 comprises a slave transceiver module 712 which is configured to generate a second interrogation signal 716 in response to said first interrogation signal 236 to induce said reply signal 238 from the wearable tag 202. Under this master-slave architecture for the tag readers, the slave tag reader 710 does not require a power source and even the control module, network module and alarm module are also not essential for the slave tag reader 710 making it cost effective. In an exemplary scenario, a wearable tag reader 210 acting as a master is tied to the right hand of a child whereas a slave wearable tag reader 710 is tied to the left hand of the child. If the child brings the left hand closer to its mouth the control module 214 of the master wearable tag reader 210 analyzes the replay signal 238 generated by the wearable tag 202 in response to the second interrogation signal 716 generated by the slave transceiver module 712 and an audible alarm is sounded by the master wearable tag reader 210. The control module 214 carries out the same steps which it would have carried out had the reply signal 238 been generated in response to the first interrogation signal 236. So, be it the right hand with the master wearable tag reader or the left hand with the slave wearable tag reader, whenever the child tries to suck a thumb/finger the alert action is initiated.

While the present invention is susceptible to various modifications and alternative user interfaces, a certain illustrated embodiment thereof is shown in the drawings and has been described above in detail. It should be understood, however, that there is no intention to limit the invention to the specific form or forms disclosed, but on the contrary, the intention is to cover all modifications, alternative user interfaces, similar features and equivalents falling within the spirit and scope of the invention, as defined in the appended claims.

What is claimed is:

1. A thumb sucking deterrent system, said system comprising:
a wearable tag configured to be removably fitted inside mouth of a person, said wearable tag comprising a tag antenna anatomically shaped to correspond to one or more sections of a person's dentition and an integrated circuit chip conductively mounted to said tag antenna; and
a wearable tag reader adapted to be worn on a part of a forearm of said person, said wearable tag reader comprising a transceiver module, a control module and an alarm module;
wherein said alarm module is configured to initiate one or more alert actions when said control module determines an actual distance between said wearable tag and said wearable tag reader to be within a predefined range based on a reply signal generated by said wearable tag in response to a first interrogation signal generated by said transceiver module.

2. The thumb sucking deterrent system of claim 1, wherein a water resistant coating is provided over at least a section of said wearable tag to electrically insulate said tag antenna and said integrated circuit chip from an outer environment.

3. The thumb sucking deterrent system of claim 1, wherein said one or more alert actions include an audible alarm emitted by a speaker disposed in said wearable tag reader and/or a visual alarm emitted by a light source disposed in said wearable tag reader.

4. The thumb sucking deterrent system of claim 1, wherein said control module is configured to adjust said predefined range and/or said first interrogation signal as per one or more instructions.

5. The thumb sucking deterrent system of claim 4, wherein said one or more instructions are received from a remote server, said control module being communicatively connected to said remote server over a network.

6. The thumb sucking deterrent system of claim 4, wherein a computing device is communicably connected to said wearable tag reader, said computing device comprising one or more physical processors programmed with computer program instructions that, when executed by said one or more physical processors, program said computing device to transmit said one or more instructions.

7. The thumb sucking deterrent system of claim 6, wherein said one or more alert actions include a notification sent to said computing device.

8. The thumb sucking deterrent system of claim 1, said system further comprises a slave wearable tag reader adapted to be worn on said part of said forearm of said person, said slave wearable tag reader comprising a slave transceiver module being configured to generate a second interrogation signal in response to said first interrogation signal to induce said reply signal.

9. The thumb sucking deterrent system of claim 1, wherein said wearable tag reader is further configured to identify said wearable tag by a unique identity.

10. The thumb sucking deterrent system of claim 1, wherein said tag antenna comprising distal ends structured to be secured to one or more teeth of said person and a frontal section configured as an RFID tag antenna.

11. A thumb sucking deterrent system, said system comprising:
a wearable tag adapted to be worn removably inside a mouth of a person, said wearable tag comprising a tag antenna anatomically shaped to correspond to one or more sections of a person's dentition and an integrated circuit chip conductively mounted to said tag antenna, said tag antenna having distal ends structured to be secured to one or more teeth of said person;
a wearable tag reader adapted to be worn on a part of a forearm of said person, said wearable tag reader comprising a transceiver module, a control module and an alarm module; and
a computing device communicably connected to said wearable tag reader, said wearable tag reader comprising one or more physical processors programmed with computer program instructions that, when executed by said one or more physical processors, program said computing device to:
configure said wearable tag reader generate a first interrogation signal as per requirement;
configure said wearable tag reader to determine an actual distance between said wearable tag and said wearable tag reader based on a reply signal generated by said wearable tag in response to said first interrogation signal generated by said transceiver module; and
initiate one or more alert actions if said actual distance is found to be falling within a predefined range.

12. The thumb sucking deterrent system of claim 11, wherein a water resistant coating is provided over at least a section of said wearable tag to electrically insulate said tag antenna and said integrated circuit chip from an outer environment.

13. The thumb sucking deterrent system of claim 11, wherein said one or more alert actions include an audible alarm emitted by a speaker disposed in said wearable tag reader and/or a visual alarm emitted by a light source disposed in said wearable tag reader.

14. The thumb sucking deterrent system of claim 11, wherein said control module is configured to adjust said predefined range and/or said first interrogation signal as per one or more instructions.

15. The thumb sucking deterrent system of claim 11, said system further comprises a slave wearable tag reader adapted to be worn on said part of said forearm of said person, said slave wearable tag reader comprising a slave transceiver module being configured to generate a second interrogation signal in response to said first interrogation signal to induce said reply signal.

16. The thumb sucking deterrent system of claim 11, wherein said wearable tag reader is further configured to identify said wearable tag by a unique identity.

17. The thumb sucking deterrent system of claim 11, wherein said one or more alert actions get recorded in said computing device.

18. The thumb sucking deterrent system of claim 11, wherein said tag antenna comprising a frontal section configured as an RFID tag antenna.
